(19) United States
(12) Patent Application Publication
(10) Pub. No.: US 2021/0078923 A1
(43) Pub. Date: Mar. 18, 2021

(54) METHOD FOR PRODUCTION OF A HALOGENATED ALKANE USING AN ABSORBER-REACTOR COMBINATION

(71) Applicant: BLUE CUBE IP LLC, Clayton, MO (US)

(72) Inventors: John D. Myers, Clayton, MO (US); Max Tirtowidjojo, Clayton, MO (US); Marc Sell, Clayton, MO (US); Thomas Luebbe, Clayton, MO (US)

(73) Assignee: BLUE CUBE IP LLC, Clayton, MO (US)

(21) Appl. No.: 17/041,321

(22) PCT Filed: Apr. 2, 2019
(86) PCT No.: PCT/US2019/025341

§ 371 Date: Sep. 24, 2020

Related U.S. Application Data
(60) Provisional application No. 62/652,072, filed on Apr. 3, 2018.

(51) Int. Cl. C07C 17/275

(52) U.S. Cl. CPC C07C 17/275

(57) ABSTRACT

The present invention provides improved processes for preparing halogenated alkanes. The processes comprise contacting at least one alkene, a halogenated alkene, or combinations thereof with a halogenated methane with at least one chlorine atom to form a liquid phase. This liquid phase is then contacted with at least one catalytic species which initiates the reaction with at least one alkene, a halogenated alkene, or combinations thereof with a halogenated methane with at least one chlorine atoms.

### FIELD OF THE INVENTION

The present disclosure generally relates to processes for preparing halogenated alkanes.

### BACKGROUND OF THE INVENTION

Halogenated alkanes are useful intermediates for many products including agricultural products, pharmaceuticals, cleaning solvents, blowing agents, solvents, gums, silicones, and refrigerants. The processes to prepare halogenated alkanes can be time consuming, moderately efficient, and lack reproducibility.

One widely known method for preparing halogenated alkanes is through a telomerization process. This process comprises contacting a halogenated methane comprising at least one chlorine atom and an alkene or halogenated alkene in the presence of a catalyst. Even though these telomerization processes are useful, these processes have variable yields, low reproducibility, large amounts of waste, and high unit manufacturing costs.

One subset of highly sought halogenated alkanes are chloropropanes especially 1,1,1,3-tetrachloropropane, 1,1,1,3,3-pentachloropropane, and 1,1,1,3,3,3-hexachloropropane which are useful intermediates for many products, including refrigerants and agricultural products. A general process for their preparation consists of reacting an alkene or a halogenated alkene, carbon tetrachloride, a trialkylphosphate, and an iron catalyst in a telomerization process. U.S. Pat. No. 4,650,914 teaches such a process where the process is conducted in batch mode, using a non-powder form of an iron and mechanical stirring. All materials are introduced into an autoclave wherein the ethylene is added to pressurize the autoclave. US 2004/0225166 teaches a similar process using a single reactor in a continuous process. Ethylene is fed into the reactor comprising carbon tetrachloride, tributylphosphate, and iron powder. The reactor is pressurized from 40 to 200 psi to maintain a concentration of ethylene. In U.S. Pat. No. 8,907,147, a similar process is described as is US 2004/0225166 wherein the ethylene is added continuously. In each of these references, ethylene is added as a gas into the reactor and must be absorbed into the liquid phase of the reaction to allow the telomerization process to proceed. Since ethylene is only partial solubility in carbon tetrachloride, the alkene or halogenated alkene is used in excess to maintain the concentration of the ethylene in the liquid phase. Similarly, iron (Fe(0)) utilized as a solid in these processes must undergo an oxidation and/or reduction to form the active, soluble catalytic species necessary to initiate the telomerization process. These processes depend on the mass transfer of the ethylene into the liquid phase of the reaction and the iron from the solid phase to liquid phase. With competing mass transfer processes occurring in the same process, one skilled in the art would find it difficult to optimize the kinetics or improve the kinetics of the process. With optimization of the mass transfer, the kinetics of the process can be therefore optimized resulting in a lower overall cost of the process.

Thus, conventional processes can be moderately efficient yet lack reproducibility, utilize expensive manufacturing equipment, have large waste factors, and provide the chlorinated propane at a higher unit manufacturing cost.

Developing a process which can prepare halogenated alkanes and chlorinated propanes where the process would exhibit high mass transfer, increased kinetics, high reproducibility, reduced amounts of waste, and reduced manufacturing costs would be desirable.

### SUMMARY OF THE INVENTION

Provided herein are processes for preparing and isolating halogenated alkanes via the reaction between at least one alkene, a halogenated alkene, or combinations thereof and a halogenated methane comprising at least one chlorine atom. The process comprises a) preparing a liquid phase in an absorber comprising contacting at least one alkene, halogenated alkene, optionally a recycle stream, at least one ligand, or combinations thereof with a halogenated methane comprising at least one chlorine atom; b) transferring at least a portion of the liquid phase from the absorber into a reaction vessel comprising a species capable of initiating the reaction of at least one alkene, halogenated alkene, or combinations thereof with a halogenated methane comprising at least one chlorine atom; and c) forming the halogenated alkane.

In another embodiment, the processes comprises a) contacting at least one alkene, halogenated alkene, or combinations thereof with a halogenated methane comprising at least one chlorine atom and optionally, a liquid recycle stream in an absorber to form a liquid phase; b) transferring at least a portion of the liquid phase from the absorber into a reaction vessel to form a reaction mixture wherein the reaction mixture comprises at least one solid metallic catalyst; at least one alkene, halogenated alkene, or combinations thereof; at least one ligand, an optional recycle stream, or combinations thereof; a halogenated methane comprising at least one chlorine atom; and c) forming a product mixture comprising the halogenated alkane, light by-products, and heavy by-products.

The at least one solid metallic catalyst (also referred to as the metallic solid catalyst or the species capable of initiating the reaction) is in the form of a powder or a fixed bed of structured or unstructured packing, or mixtures thereof. This applies to all aspects and embodiments disclosed herein.

The metallic solid catalyst is present in the reaction vessel and not in the absorber. The optimization of the gas/liquid mass transfer can occur in the absorber while the solid/liquid mass transfer can occur in the reaction vessel. Therefore, with each optimization in the absorber and reaction vessel, the gas/liquid mass transfer of the process can be optimized and the reaction kinetics can be optimized independently. This applies to all aspects and embodiments disclosed herein.

In another aspect, disclosed herein are processes for the preparation of 1,1,1,3-tetrachloropropane (250 FB). The processes comprise a) preparing a liquid phase in an absorber comprising contacting ethylene, carbon tetrachloride, and at least one ligand; b) transferring at least a portion of the liquid phase from the absorber into a reaction vessel comprising a species capable of initiating the reaction between ethylene and carbon tetrachloride; and c) forming 1,1,1,3-tetrachloropropane (250 FB).

In an additional aspect, disclosed herein are processes for the preparation of 1,1,1,3,3-pentachloropropane (240 FA). The processes comprise a) preparing a liquid phase in an absorber comprising contacting vinyl chloride, carbon tetrachloride, and at least one ligand; b) transferring at least a portion of the liquid phase from the absorber into a reaction vessel comprising a species capable of initiating the reaction between vinyl chloride and carbon tetrachloride; and c) forming 1,1,1,3,3-pentachloropropane (240 FA).

In still another aspect, disclosed herein are processes for the preparation of 1,1,1,3,3,3-hexachloropropane (111333). The processes comprise a) preparing a liquid phase in an absorber comprising contacting vinylidene chloride, carbon tetrachloride, and at least one ligand; b) transferring at least a portion of the liquid phase from the absorber into a reaction vessel comprising a species capable of initiating the reaction between vinylidene chloride and carbon tetrachloride; and c) forming 1,1,1,3,3,3-hexachloropropane.

These processes have been shown to improve the overall yield, purity, cycle time, selectivity, reduction of waste, lower unit manufacturing cost, and through-put. They also reduce the overall cost, when compared to other conventional processes. In an additional aspect, the separated reactants and heavy by-products are recycled back to the process (preferably to the inlet of the absorber, the reaction vessel or both) to provide additional efficiencies and cost reductions.

Other features and iterations of the invention are described in more detail below.

### DETAILED DESCRIPTION OF THE INVENTION

Disclosed herein are processes for the preparation of halogenated alkanes. In general, the processes comprise a reaction between an alkene, a halogenated alkene, or combinations thereof and a halogenated methane comprising at least one chlorine atom under conditions detailed below.

In all embodiments, a liquid phase is prepared by contacting at least one alkene, halogenated alkene, or combinations thereof, a halogenated methane comprising at least one chlorine atom, and at least one ligand in an absorber. The liquid phase in the absorber contains high levels of the at least one alkene, halogenated alkene, or combinations thereof in the halogenated methane comprising at least one chlorine atom. At least a portion of the liquid phase from the absorber is transferred to a reaction vessel comprising a species capable of initiating the reaction of the at least one alkene, halogenated alkene, or combinations thereof with a halogenated methane comprising at least one chlorine atom under conditions described below. Thus, producing the halogenated alkane. The species capable of initiating the reaction of at least one alkene, halogenated alkene, or combinations thereof with a halogenated methane comprising at least one chlorine atom comprises at least a metallic solid catalyst in the form of a fixed bed of structured or unstructured packing or a powder.

### (I) Processes for the Production of Halogenated Alkanes

One aspect of the present disclosure encompasses processes for the preparation of halogenated alkanes. These processes comprise forming a liquid phase in an absorber by contacting at least one alkene, halogenated alkene, or combinations thereof, a halogenated methane comprising at least one chlorine atom, and at least one ligand. At least a portion of the liquid phase from the absorber is transferred to the reaction vessel which comprises a species capable of initiating the reaction of the at least one alkene, halogenated alkene, or combinations thereof with the halogenated methane comprising at least one chlorine atom. The halogenated alkane is formed. By preparing the liquid phase in the absorber comprising at least one alkene, halogenated alkene, or combinations thereof, the halogenated methane comprising at least one chlorine atom, and at least one ligand, a high concentration of the at least one alkene, halogenated alkene, or combinations thereof is prepared and the mass transfer and kinetics of the process is increased. Therefore as a result, the output and kinetics of the process are maintained at a high rate.

(a) Liquid Phase in the Absorber

The process commences by preparing a liquid phase in the absorber. Initially, at least one alkene, halogenated alkene, or combinations thereof is contacted with a halogenated methane comprising at least one chlorine atom. The absorber does not contain any catalytic species that is solid or metallic, where metallic is understood to mean a zero valent metal.

(i) Alkene, Halogenated Alkene, or Combinations Thereof

A wide variety of alkenes, halogenated alkenes, or combinations thereof may be used in the process. As appreciated by the skilled artisan, the alkene, halogenated alkene, or combinations thereof may be introduced in the absorber as a liquid or a gas wherein the alkene, halogenated alkene, or combinations thereof may be at least partially soluble in the liquid phase. In various embodiments, the at least one alkene, halogenated alkene, or combinations thereof may be introduced above the surface of the liquid phase or below the surface of the liquid phase through a port in the absorber. The alkene, a halogenated alkene, or combination thereof is introduced into the absorber to prepare a high concentration of the alkene, halogenated alkene, or combinations thereof in the halogenated methane comprising at least one chlorine atom and/or to maintain a pressure within the absorber.

Generally, the at least one alkene, halogenated alkene, or combinations thereof comprise between 2 and 5 carbon atoms. Non-limiting examples of alkenes may be ethylene, propylene, 1-butene, 2-butene, isobutene, 1-pentene, 2-pentene, 3-pentene, 2-methyl-2-butene, 2-methyl-1-butene, and 3-methyl-1-butene. Non-limiting examples of halogenated alkenes may be vinyl chloride, vinyl bromide, vinyl fluoride, allyl chloride, allyl fluoride, 1-chloro-2-butene, 1-fluoro-2 butene, 3-chloro-1-butene, 3-fluoro-1-butene, 3-chloro-1-pentene, 3-fluoro-1-pentene, and combinations thereof. In one embodiment, the alkene comprises ethylene, propylene, 1-butene, 2-butene, isobutylene, or combinations thereof. In one preferred embodiment, the alkene comprises ethylene. In another embodiment, the halogenated alkene is vinyl chloride, vinylidene chloride, trichloroethylene, perchloroethylene, 1,2,3-trichloropropene, 1,1,3-trichloropropene, 3,3,3-trichloropropene, or combinations thereof. In one embodiment, the halogenated alkene comprises 1,1,3-trichloropropene, 3,3,3-trichloropropene, or combinations thereof. In another embodiment, the halogenated alkene comprises vinyl chloride or vinylidene chloride. In a different embodiment, the halogenated alkene comprises vinyl chloride.

(ii) Halogenated Methane Comprising at Least One Chlorine Atom

A wide variety of halogenated methanes comprising at least one chlorine atom may be used in this process. Non-limiting examples of halogenated methane comprising at least one chlorine atom include methyl chloride, methylene chloride, chloroform, carbon tetrachloride, chlorofluoromethane, dichloromonofluoromethane, trichlorofluoromethane, difluorochloromethane, trifluorochloromethane, bromochloromethane, dibromochloromethane, tribromochloromethane, chloroiodomethane, chlorodiiodomethane, chlorotriiodomethane, bromochlorofluoromethane, bromochlorodifluoromethane, chlorodibromofluoromethane, bromochlorofluoroiodomethane, bromochlorodiiodomethane, and combinations thereof. In one preferred embodiment, the halogenated methane comprising at least one chlorine atom is carbon tetrachloride.

In general, the halogenated methane comprising at least one chlorine atom is used in excess in the fresh material feed, but substoichiometric amounts are acceptable. Generally, the molar ratio of the halogenated methane comprising at least one chlorine atom to an alkene, a halogenated alkene, or combinations thereof may range from 0.1:1 to about 100:1. In various embodiments, the molar ratio of the halogenated methane comprising at least one chlorine atom to an alkene, a halogenated alkene, or combinations thereof may range from 0.1:1 to about 100:1, from 0.5:1 to about 75:1, from 1:1 to about 10:1, or from 1.2:1 to about 5:1. In various embodiments, the molar ratio of the halogenated methane comprising at least one chlorine atom to an alkene, a halogenated alkene, or combinations thereof may range from 1.2:1 to about 2:1. The halogenated methane comprising at least one chlorine atom and the at least one alkene, a halogenated alkene, or combinations thereof are essentially dry, i.e., it has a water content of the below 1000 ppm. Lower water concentrations are preferred, but not required.

(iii) The at Least One Ligand

In various embodiments, at least one ligand is used in the process. Without wishing to be bound to a theory, it is believed that the ligand complexes with the transition metal to form a ligand transition metal complex, which is soluble in the reaction media.

In one embodiment, the at least one ligand comprises at least one trialkylphosphate, at least one trialkylphosphite, an alkyl nitrile, or combinations thereof. In one embodiment, the ligand is a phosphorus containing compound. Examples of phosphorus containing compound may include trialkylphosphates, trialkylphosphites, or combinations thereof. Suitable non-limiting examples of trialkylphosphates include triethylphosphate, tripropylphosphate, triisopropylphosphate and, tributylphosphate. Examples of trialkylphosphites include trimethylphosphite, triethylphosphite, tripropylphosphite, triisopropylphosphite, tributylphosphite, and tri-tert-butylphosphite. In another embodiment, the ligand is an alkyl nitrile. Non-limiting examples of alkyl nitriles include propanenitrile, butanenitrile, pentanenitrile, hexanenitrile, or combinations thereof. In one preferred embodiment, the ligand is a trialkylphosphate. More preferably, the ligand is tributylphosphate.

(iv) Reaction Conditions

A portion of the at least one alkene, halogenated alkene, or combinations thereof may be released from the liquid phase as a gas into the headspace. Thus, gas can be present in the reactor. There are many methods to stir the contents of the absorber and/or provide increased gas absorption into the liquid phase. Stirring provides a high concentration of the at least one alkene, halogenated alkene, or combinations thereof in the halogenated methane comprising at least one chlorine atom. In various embodiments, these methods simply mix the liquid phase of the reaction mixture in the absorber. In other embodiments, the method not only mixes the liquid phase of the reaction mixture but also provide increased gas absorption into the liquid phase of the reaction mixture. In still another embodiment, the method provides increased absorption of the gas phase into the liquid phase of the reaction mixture of the absorber. Non-limiting methods to adequately stir the liquid phase contents of the absorber may be jet stirring, impellers, baffles in the absorber, or combinations thereof. Non-limiting examples of methods to mix the contents of the absorber and provide increased gas absorption into the liquid phase of the reaction mixture include jet stirring using at least one eductor, jet stirring comprising at least one nozzle and at least one eductor, jet stirring wherein jet stirring comprises at least one nozzle is directed through the gas phase into the liquid phase, specially designed impellers that create adequate gas absorption into the liquid phase, absorber with specially designed baffles, and combinations thereof. A non-limiting example of a method to provide increased absorption of the gas phase into the liquid phase of an absorber is a spray nozzle, wherein the liquid phase is pumped through the spray nozzle into the gas phase resulting in absorption of the gas into the liquid spray. In one embodiment, the absorber comprises a spray tower or packing to facilitate the absorption and mixing of the reactants. The absorber may be a packed column, which may comprise a fixed bed of structured or unstructured packing, or mixtures thereof. The exact shape and size of the absorber is variable and depends, for example, on the amount of material being produced, the pressure in the reaction system, and the nature of the reagents. The purpose of the absorber is to increase the contact of and to facilitate the mixing of the reactants. These methods can be used to maintain the kinetics of the process.

Jet mixing utilizing at least one nozzle withdraws a portion of the liquid phase of the reaction mixture from the absorber and pumps the liquid phase back into the absorber through at least one nozzle. This creates turbulence in the liquid phase and increases mixing. The at least one nozzle may be positioned below the surface of the liquid phase, at the surface of the liquid phase or directed through the gas phase into the liquid phase.

Jet mixing utilizing at least one eductor withdraws a portion of the liquid phase of the reaction mixture from the reactor and pumps the liquid phase back into the reactor through at least one gas educting nozzle. The eductor nozzle provides suction in the eductor which pulls gas from the gas phase of the reaction mixture, mixes the gas with the circulated liquid phase, and returns the resulting mixture of liquid and gas back into the liquid phase of the absorber, where the liquid had increased absorption of the gas as compared to the circulated liquid phase. When the flow from the eductor nozzle is directed towards the liquid phase of the reaction mixture, increased gas absorption of the gas in the liquid phase and increased turbulence of the reaction mixture result.

Jet mixing may also utilize at least one nozzle and at least one eductor. In this configuration, as described above, not only increased turbulence in the reaction mixture but also increased gas absorption of the gas into the liquid phase may be realized.

The use of a spray nozzle may also be utilized. Using a spray nozzle, the liquid phase is pumped through the spray nozzle producing droplets of the liquid phase from the reaction mixture. These droplets may be discharged into the gas phase, where they absorb at least some of the gas phase. The droplets are then reincorporated into the liquid phase of the reaction mixture, thereby increasing the amount of gas dissolved in the liquid phase of the reaction mixture.

In other embodiments, a draft tube may be utilized in the process. The draft tube provides an internal recirculation of the reaction mixture within the absorber. The circulation may be induced by energy from the at least one liquid jets, from the at least one gas educting nozzle, from rising gas bubbles within the reactor, or a combination thereof.

As appreciated by the skilled artisan, one or more of the methods described above may be utilized in the process.

In general, the process for the preparation of halogenated alkanes will be conducted to maintain the temperature from about 80° C. to about 130° C. using an internal or external heat exchanger. In various embodiments, the temperature of the reaction may be maintained from about 80° C. to about 130° C., from 85° C. to about 125° C., from 90° C. to about 120° C., or from about 95° C. to about 110° C.

Generally, the process may be conducted at a pressure of about atmospheric pressure (˜14.7 psi) to about 400 psi so the amount of the gases and liquid are in suitable quantities so the reaction may proceed and maintain the kinetics of the process. In various embodiments, the pressure of the process may be from about atmospheric pressure to about 400 psi, from about 20 psi to about 380 psi, from about 40 psi to about 300 psi, from about 80 psi to about 200 psi, or from 100 psi to about 120 psi.

(b) Preparation of the Halogenated Alkane

The next step in the process comprises transferring a portion of the liquid phase from the absorber to a reaction vessel. The reaction vessel comprises a species capable of initiating the reaction of the at least one alkene, halogenated alkene, or combinations thereof with the halogenated methane comprising at least one chlorine atom and is contacted with the liquid phase from the absorber which forms the halogenated alkane under conditions detailed below. The species capable of initiating the reaction of at least one alkene, halogenated alkene, or combinations thereof with a halogenated methane comprising at least one chlorine atom comprises at least one metallic solid catalyst in the form of fixed bed of structured or unstructured packing or a powder. In various embodiments, the process may be conducted in batch or continuous mode.

(i) At Least One Metallic Solid Catalyst

A wide variety of at least one metallic solid catalyst as a source of the catalytic species may be used in the process. In some embodiments, the catalytic species of the at least one metallic solid catalyst may comprise a transition metal. As used herein, the term “transition metal” refers to a transition metal element, a transition metal containing alloy, a transition metal containing compound, or combinations thereof. Non limiting examples of transition metals in the at least catalytic species may be selected from the group consisting of aluminum, bismuth, chromium, cobalt, copper, gallium, gold, indium, iron, lead, magnesium, manganese, mercury, nickel, platinum, palladium, rhodium, samarium, scandium, silver, titanium, tin, zinc, zirconium, and combinations thereof. In a preferred embodiment, the catalytic species may comprise a solid transition metal selected from the group consisting of iron, copper, and combinations thereof.

Non-limiting examples of metal containing alloys useful in the process may be an alloy of aluminum, an alloy of bismuth, an alloy of chromium, an alloy of cobalt, an alloy of copper, an alloy of gallium, an alloy of gold, an alloy of indium, an alloy of iron, an alloy of lead, an alloy of magnesium, an alloy of manganese, an alloy of mercury, an alloy of nickel, an alloy of platinum, an alloy of palladium, an alloy of rhodium, an alloy of samarium, an alloy of scandium, an alloy of silver, an alloy of titanium, an alloy of tin, an alloy of zinc, an alloy of zirconium, and combinations thereof. Non-limiting common names for these alloys may be Al—Li, Alnico, Birmabright, duraluminum, hiduminum, hydroalium, magnalium, Y alloy, nichrome, stellite, ultimet, vitallium, various alloys of brass various alloys of brass, bronze, constantin, Corinthian bronze, cunife, cupronickel, cymbal metals, electrum, haptizon, manganin, nickel silver, Nordic gold, tumbaga, crown gold, colored gold, electrum, rhodite, rose gold, tumbaga, white gold, cast iron, pig iron, Damascus steel, wrought iron, anthracite iron, wootz steel, carbon steel, crucible steel, blister steel, alnico, alumel, brightray, chromel, cupronickel, ferronickel, German silver, Inconel, monel metal, nichrome, nickel-carbon. Nicrosil, nitinol, permalloy, supermalloy, 6al-4v, beta C, gum metal, titanium gold, Babbitt, britannium, pewter, solder, terne, white metal, sterling silver, zamak, zircaloy, or combinations thereof. In one embodiment, the at least one metallic solid catalyst comprises a metal, a metal powder, an alloy of a metal, or combinations thereof. In a preferred embodiment, at least one metallic solid catalyst as a source of the catalytic species may be iron metal, copper metal, an iron containing compound, a copper containing compound, an alloy of iron, an alloy of copper, or combinations thereof, may be in various forms. In one embodiment, the metal comprises iron metal, an iron containing compound, an iron containing alloy, or combinations of two or more thereof. In another embodiment, the metal comprises copper metal, an iron containing compound, an iron containing alloy, or combinations of two or more thereof.

Generally, at least one metallic solid catalyst as a source of the catalytic species may be in various forms or configuration. Non-limiting examples of the forms or configuration of at least one metallic solid catalyst may be a packing, an unstructured packing, a foil, a sheet, a screen, a wool, a wire, a ball, a plate, a pipe, a rod, a bar, or a powder. In other embodiments, the iron or copper may be mobilized on the surface of a support. Non-limiting examples of suitable supports may be alumina, silica, silica gel, diatomaceous earth, carbon and clay. Further examples include copper on alumina, copper on silica, iron on carbon, iron on diatomaceous earth, and iron on clay.

As appreciated by the skilled artisan, the catalyst, once in the process, may undergo oxidation and/or reduction to produce an activated catalytic species in various oxidation states. The oxidation state of these active iron catalytic species may vary, and may be for examples (0), (I), (II), and (III). In one aspect, the active iron catalyst may in the Fe(0) or Fe(I) oxidation state. In another aspect, the active iron catalyst may be Fe(II). In still another aspect, the active iron catalyst may be in the Fe(III) oxidation state. In an additional aspect, the active iron catalyst may comprise a mixture of Fe(I) and Fe(II). In still another aspect, the active iron catalyst may comprise a mixture of Fe(I) and Fe(III) oxidation states. In yet another aspect, the active iron catalyst may be in the Fe(II) and Fe(III) oxidation states. In one aspect, the active iron catalyst may in the Fe(I), Fe(II) and Fe(III) oxidation states. In another aspect, the active iron catalyst may in the Fe(I), Fe(II) and Fe(III) oxidation states. In still another embodiment, an electrochemical cell may be utilized to adjust the ratio of Fe(I), Fe(II), and Fe(III) in the process. The oxidation state of these active copper catalytic species may vary, and may be for examples (I) and (II). In one aspect, the active copper catalyst may in the Cu(I) oxidation state. In another aspect, the active copper catalyst may be Cu(II). In one embodiment, the active copper catalyst may comprise a mixture of Cu(0), Cu(I) and Cu(II). In an additional aspect, the active copper catalyst may comprise a mixture of Cu(I) and Cu(II). In still another aspect, an electrochemical cell may be utilized to adjust the ratio of Cu(I), and Cu(II) in the process.

In still another embodiment, the at least one metallic solid as a source of the catalytic species in a continuous reactor may be part of at least one fixed catalyst bed. In still another embodiment, the at least one metallic solid in a continuous reactor may be part of at least one cartridge. In still another embodiment, the at least one metallic solid may be part of a structured or un-structured packing where the at least one catalyst is a part of the packing or un-structured packing. Using a fixed catalyst bed, a cartridge, structured packing, or unstructured packing, the catalytic species may be contained and easily replaced when consumed. Non-limiting examples of structured and unstructured packing may be any metallic form for random packing, or combinations thereof. In an embodiment, the packing comprises Raschig™ rings, pall rings, saddles, cylinders, spheres, mesh, Koch Sulzer™ packing, bars, nails, random shapes, or combinations thereof.

Generally, the porosity of the at least one metallic solid is less than 0.95. In various embodiments, the porosity of the at least one catalytic species is less than 0.95, less than 0.8, less than 0.5, less than 0.3, or less than 0.1. Further, the porosity of may range from 0.1 to about 0.95, from 0.3 to about 0.8, or from 0.4 to about 0.6.

The ratio of the surface area of the catalyst to the halogenated methane comprising at least one chlorine atom is at least 0.1 cm²/(g/hr). In various embodiments, the ratio of the surface area of the catalyst to the halogenated methane comprising at least one chlorine atom is at least 0.1 cm²/(g/hr), at least 0.5 cm²/(g/hr), at least 1.0 cm²/(g/hr), at least 1.5 cm²/(g/hr), or at least 2.0 cm²/(g/hr).

In general, the molar ratio of the dissolved elemental metal to the ligand may range from 1:1 to about 1:1000. In various embodiments, the molar ratio of the dissolved elemental metal to the ligand may range from 1:1 to about 1:1000, from 1:1 to about 1:500, from 1:1 to about 1:100, or from 1:1 to about 1:10. In one preferred embodiment, the molar ratio of the dissolved elemental metal to the ligand may range from 1:1.5 to about 1:3.

(ii) optional halogenated methane comprising at least one chlorine atom.

In various embodiments, the catalytic species may further comprise a halogenated methane comprising at least one chlorine atom. In other embodiments, the catalytic species may be devoid of the halogenated methane comprising at least one chlorine atom.

(iii) transferring of the liquid phase from the absorber to the reaction vessel.

At least a portion of the liquid phase from the absorber is transferred to the reaction vessel. In one aspect, when starting the process, the reaction vessel contains the at least one metallic solid catalyst as a source of the catalytic species and the halogenated methane comprising at least one chlorine atom. In another aspect, the second reaction vessel only comprises the at least one metallic solid catalyst. In a preferred embodiment, when starting the process, the reaction vessel contains the at least one metallic solid catalyst and the halogenated methane comprising at least one chlorine atom.

(iv) Reaction Conditions

As appreciated by the skilled artisan, there are many methods to stir the contents of reaction vessel comprising the liquid phase from the absorber and the liquid phase from the reaction vessel, and to provide mixing with the at least one metallic solid catalyst. These methods would provide increased interaction between the liquid phases and at least one metallic solid catalyst. Non-limiting methods to adequately stir the liquid phase contents of the reactor may be jet stirring, impellers, baffles in the reactor, or combinations thereof.

The importance of mixing is to maximize solid-liquid mass-transfer by maximizing contact between the liquid phase and the at least one metallic solid catalyst. Therefore, the type of mixing depends on the form of the at least one metallic solid catalyst. For example, when the at least one metallic solid catalyst is in powder form, an impeller with or without baffles aids in suspending, mixing, and fluidizing of the at least one metallic catalyst to maximize contact area and provide fresh liquid contact with the powder.

In another embodiment, when the at least one solid metallic catalyst is in the form of a fixed bed, then the liquid phase is fed directly into the fixed bed from one end of the fixed bed and exit of the other end. The fixed bed may be contained within a cylindrical or tubular container. Generally, the L/D (length/diameter) of the cylindrical or tubular container may be greater than 1. In various embodiments, the L/D (length/diameter) of the cylindrical or tubular container may be greater than 1, greater than 2, greater than 4, greater than 6, or greater than 8. The residence time and velocity of the fluid in the fixed bed may be varied by recycling a portion of the fixed bed reactor effluent back to the inlet. The fixed bed reactor temperature may also be independently varied from the absorber temperature by heat exchanging the reactor recycle stream. The fixed bed temperature may also be controlled by including internal heat exchanger such as the use of multitube exchanger.

As appreciated by the skilled artisan, at least one of the methods or a combination of these may be utilized in the process.

In general, the process for the preparation of halogenated alkanes will be conducted to maintain the temperature from about 80° C. to about 130° C. using an internal or external heat exchanger. In various embodiments, the temperature of the reaction may be maintained from about 80° C. to about 130° C., from 85° C. to about 125° C., from 90° C. to about 120° C., or from about 95° C. to about 110° C. In one embodiment, the temperature within the absorber and the reaction vessel are the same. In another embodiment, the temperature within the absorber and the reaction vessel are different.

Generally, the process may be conducted at a pressure of about atmospheric pressure (˜14.7 psi) to about 200 psi so the amount of the gases and liquid are in suitable quantities so the reaction may proceed and maintain the kinetics of the process. In various embodiments, the pressure of the process may be from about atmospheric pressure (˜14.7 psi) to about 200 psi, from about 20 psi to about 180 psi, from about 40 psi to about 160 psi, from about 80 psi to about 140 psi, or from 100 psi to about 120 psi. In one embodiment, the pressure within the absorber and the reaction vessel are the same. In another embodiment, the pressure within the absorber and the reaction vessel are different.

Generally, the reaction is allowed to proceed for a sufficient period of time until the reaction is complete, as determined by any method known to one skilled in the art, such as chromatography (e.g., GC-gas chromatography). The duration of the reaction may range from about 5 minutes to about 16 hours. In some embodiments, the duration of the reaction may range from about 5 minutes to about 16 hours, from about 1 hour to about 12 hours, from about 2 hours to about 10 hours, from about 4 hours to about 8 hours, or from about 5 hours to about 7 hours.

(c) Output from Process

The process, as outlined above, produces the halogenated alkane(s), light by-products and heavy by-products. In general, the process produces the halogenated alkanes in at least 50 weight percent (wt %) in the liquid phase of the reactor. In various embodiments, the halogenated alkane is produced in at least 50 wt %, in at least 60 wt %, in at least 70 wt %, in at least 80 wt %, in at least 90 wt %, in at least 95 wt %, or in at least 99 wt % in the liquid phase of the reactor.

In general, the halogenated methane comprising at least one chlorine atom is converted into the halogenated alkane in at least 50%. In various embodiments, the % conversion of the halogenated methane comprising at least one chlorine atom into the halogenated alkane is at least 50%, in at least 60%, in at least 70%, in at least 80%, in at least 90%, or at least 95%.

Generally, the process produces halogenated alkanes, light by-products, and heavy by-products. These heavy by-products are produced in less than 5 weight % in the entire product distribution. In various embodiments, these heavy by-products may be less than 4 weight %, less than 3 weight %, less than 2 weight %, or less than 1 weight %.

Generally, the halogenated alkane is a chlorinated alkane comprising between 3 and 4 carbons and between 2 and 8 chlorine atoms. Non-limiting examples of chlorinated propanes which may be prepared by this process may be 1,1,1,3-tetrachloropropane (250 FB); 1,1,1,3,3-pentachloropropane (240 FA); 1,1,1,3,3,3-hexachloropropane (111333); or combinations thereof.

In a preferred embodiment, the halogenated alkane is a chlorinated alkane wherein the chlorinated alkane is 1,1,1,3-tetrachloropropane (250 FB); 1,1,1,3,3-pentachloropropane (240 FA); or 1,1,1,3,3,3-hexachloropropane (111333).

In an exemplary embodiment, the following chlorinated propanes and chlorinated butanes may be prepared by the process disclosed herein as shown in the below scheme.

Chlorinated Propanes

CH₂═CH₂+CCl₄=>CCl₃—CH₂—CH₂Cl (1113-250 FB)

CHCl=CH₂+CCl₄=>CCl₃—CH₂—CHCl₂ (11133-240 FA)

CCl₂=CH₂+CCl₄=>CCl₃—CH₂—CCl₃ (111333)

### (II) Separation of the Halogenated Alkane and Recycle Streams

The next step in the process comprises separating purified halogenated alkane from the reaction mixture effluent stream, which comprises halogenated alkane, a halogenated methane comprising at least one chlorine atom, an alkene, halogenated alkene, or combinations thereof, the at least one ligand, at least one metallic catalytic species, heavy by-products, and light by-products through at least one separator and alternatively a second separator in order to isolate the halogenated alkane in the desired yield and/or purity. In various embodiments, at least one of the first separator and the second separator may be a distillation column or a multistage distillation column. Additionally, the at least one of the first separator and the second separator may further comprise a reboiler, a bottom stage, or a combination thereof. Various distillation columns may be used in this capacity. In one embodiment, a side draw column or a distillation column which provides an outlet stream from an intermediate stage or a dividing wall column (dividing wall column (DWC) is a single shell, fully thermally coupled distillation column capable of separating mixtures of three or more components into high purity products, i.e., product effluent streams) may be used as a separator. Product effluent streams are purer than the reaction mixture effluent stream, because they are generated by treating the reaction mixture effluent stream and removing at least some undesired components. A portion of various product effluent streams or a portion of the reaction mixture effluent produced by the process are optionally recycled back into the reactor to provide increased kinetics, increased efficiencies, reduced overall cost of the process, increased selectivity of the desired halogenated alkane, and increased yield of the desired halogenated alkane. In one embodiment, at least one product effluent stream, a portion of the reaction mixture effluent stream, or combinations thereof are sent to the absorber or the reaction vessel, wherein the temperature of the at least one product effluent stream, a portion of the reaction mixture effluent stream, or combinations thereof is maintained with a heat exchanger. In an embodiment, at least a portion of the reaction mixture effluent stream is treated to remove light by-products, heavy by-products, or combinations thereof from the halogenated alkane. If desired, at least a portion of the light by-products is recycled to the absorber, at least a portion of the heavy by-products is recycled to the reaction vessel, or both at least a portion of both the light by-products and heavy by-products are recycled.

Separating the purified halogenated alkane from the reaction mixture effluent from the reactor would produce at least two, but typically three product effluent streams. In various embodiments, separating the purified chlorinated alkane may produce four, five, or more product effluent streams depending on the separation device utilized. As an example, the separation of the chlorinated alkane from the reaction mixture effluent stream into three product effluent streams is described below.

The process utilizing one separator commences by transferring a portion of the reaction mixture effluent from the reaction vessel into a separator. In this operation, at least a portion of the reaction mixture effluent stream is separated into three distinct product effluent streams, product effluent stream (a), (b), and (c). Product effluent stream (a), as an overhead stream, comprises light by-products, hydrogen chloride, an alkene, halogenated alkene, or combinations thereof, and the halogenated methane comprising at least one chlorine atom; product effluent stream (b) comprising the halogenated alkane; and product effluent stream (c), as a bottom stream, comprising heavy by-products, the at least one ligand, and the at least one catalytic species.

In another embodiment, product effluent stream (a) may be transferred into a second separator producing two distinct product effluent streams (d) and (e). Product effluent stream (d) comprising hydrogen chloride may be captured or recycled to another process since hydrogen chloride is a valuable commercial material. A portion of product effluent stream (e) comprising light by-products, an alkene, halogenated alkene, or combinations thereof, and the halogenated methane comprising at least one chlorine atom may be recycled to the absorber or used in another process.

In yet another embodiment, product effluent stream (b) comprising the halogenated alkane may be transferred into an additional separation device to achieve the desired purity of the halogenated alkane.

In still another embodiment, at least a portion of product effluent stream (c) comprising heavy by-products, the at least one ligand, and the at least one active catalytic species may be recycled to the reaction vessel or used in another process.

In various embodiments, at least a portion of product effluent streams (c) and/or (e) may be recycled back into the reaction vessel or mixed with fresh feed before being recycled back into the reaction vessel. These streams may also be fed into another process to produce other products. These steps may be performed in any order to improve the efficiency, reduce the cost, reduce contaminants, and increase through-put of the process.

In another embodiment, at least a portion of product effluent streams (c) and/or (e) may be mixed with fresh material feeds before being recycled back into the absorber in batch mode or continuous mode, where the fresh material feeds comprise a halogenated methane comprising at least one chlorine atom, an alkene, halogenated alkene, or combinations thereof, the at least one ligand, or combinations thereof. The fresh material feed may be added to the absorber, reaction vessel, or combinations thereof. In various embodiments, the recycle product effluent streams and fresh material feed streams may be introduced into the absorber separately or mixed together before entering the process. The introduction of these fresh material feeds into the absorber or mixing the recycle product effluent streams with fresh feeds increases the efficiency of the process, reduces the overall cost, maintains the kinetics, increase the through-put, and reduces the by-products produced by the process. Optionally, a portion of the fresh material feed may be added directly into the reaction vessel bypassing the absorber. Additionally, this fresh material feed may be premixed with the liquid phase from the absorber or a product effluent stream before being added to the reactor. Also, the fresh material feed may be directly added into the reactor. The amounts of the recycle product effluent streams or fresh material feed streams added to the reactor may be the same or different. One way to measure the amount of the recycle product effluent streams or fresh material feed streams being added to the reactor is to identify the mass flow of each of these streams. The product effluent streams being recycled to the reactor and/or the absorber have a recycle product effluent mass flow, while the fresh material feed streams being added to the reactor has a fresh material feed mass flow. Mass flows may be measured using methods known in the art.

Generally, the mass ratio of the product effluent stream mass flow being recycled to the fresh material feed mass flow is adjusted to maintain the conversion of the process and/or maintain the kinetics of the process.

In yet another embodiment, the active catalytic species may be separated from the product stream by means of extraction. This extraction, using water or another polar solvent, may remove deactivated catalyst. The extraction may separate the active catalytic species which may be introduced back into the reaction vessel or other downstream processes. Using the extraction processes defined above may provide added efficiency to the process in respect to overall cost.

Product effluent streams (b) comprising the halogenated alkane produced in the process may have a yield of at least about 20%. In various embodiments, the product effluent stream (b) comprising halogenated alkane produced in the process may have a yield of at least about 20%, at least about 50%, at least about 70%, at least about 75%, at least about 80%, at least about 85%, at least about 90%, at least about 95%, or at least about 99%.

The halogenated alkane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (III) Preferred Embodiments: 1,1,1,3-Tetrachioropropane

(a) Process for the Preparation of 1,1,1,3-Tetrachloropropane

One aspect of the present disclosure encompasses processes for the preparation of 1,1,1,3-tetrachloropropane. The process commences by preparing a liquid phase in the absorber comprising contacting ethylene, carbon tetrachloride, and the at least one ligand which does not contain any catalytic species. The liquid phase from the absorber is transferred to the reaction vessel comprising a species capable of initiating the reaction of ethylene with carbon tetrachloride, the at least one ligand, and optionally carbon tetrachloride under the reaction conditions described above. The species capable of initiating the reaction of at least one alkene, halogenated alkene, or combinations thereof with a halogenated methane comprising at least one chlorine atom comprises at least one metallic solid catalyst in the form of fixed bed of structured or unstructured packing or a powder. The at least one metallic solid catalyst utilized in the reaction vessel is described in Section (I)(b)(i). The optional ligand in the absorber is described in Section (I)(a)(iii).

(b) Reaction Conditions

The reaction conditions for the preparation of the liquid phase in the absorber are described above in Section (I)(a)(iv). The reaction conditions for the preparation of the liquid phase in the reaction vessel is described (I)(b)(iv).

(c) Output from Process

In a preferred embodiment, the process produces 1,1,1,3-tetrachloropropane. In general, the process produces 1,1,1,3-tetrachloropropane in at least 50 weight percent (wt %) in the liquid phase of the reactor. In various embodiments, 1,1,1,3-tetrachloropropane is produced in at least 50 wt %, in at least 60 wt %, in at least 70 wt %, in at least 80 wt %, in at least 90 wt %, in at least 95 wt %, or in at least 99 wt % in the liquid phase of the reactor.

In general, carbon tetrachloride is converted into 1,1,1,3-tetrachloropropane in at least 50% conversion. In various embodiments, the % conversion of carbon tetrachloride into 1,1,1,3-tetrachloropropane is at least 50%, in at least 60%, in at least 70%, in at least 80%, in at least 90%, or at least 95%.

Generally, the process produces 1,1,1,3-tetrachloropropane, light by-products and heavy by-products. These heavy by-products are produced in less than 5 weight % in the entire product distribution. In various embodiments, these heavy by-products may be less than 4 weight %, less than 3 weight %, less than 2 weight %, or less than 1 weight %.

(d) Separation of 1,1,1,3-tetrachloropropane.

The separation of 1,1,1,3-tetrachloropropane and the recycle streams is described above in Section (II).

Product effluent stream (b) comprising the 1,1,1,3-tetrachloropropane produced in the process may have a yield of at least about 20%. In various embodiments, the product effluent stream (b) comprising 1,1,1,3-tetrachloropropane produced in the process may have a yield of at least about 30%, at least about 50%, at least about 70%, at least about 75%, at least about 80%, at least about 85%, at least about 90%, at least about 95%, or at least about 99%.

The 1,1,1,3-tetrachloropropane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (IV) Preferred Embodiments: 1,1,1,3,3-Pentachioropropane

(a) Process for the Preparation of 1,1,1,3,3-Pentachloropropane

One aspect of the present disclosure encompasses processes for the preparation of 1,1,1,3,3-pentachloropropane. The process commences by preparing a liquid phase in the absorber comprising contacting vinyl chloride, carbon tetrachloride, and at least one ligand, which does not contain any catalytic species. The liquid phase from the absorber is transferred to the reaction vessel comprising at least one metallic solid catalyst in the form of fixed bed of structured or unstructured packing or a powder capable of initiating the reaction of vinyl chloride with carbon tetrachloride, at least one ligand, and optionally carbon tetrachloride under the reaction conditions described above. To be clear, species capable of initiating the reaction of vinyl chloride with a halogenated methane comprising at least one chlorine atom are present in the reaction vessel and not in the absorber. The at least one metallic solid catalyst utilized in the reaction vessel is described in Section (I)(b)(i). The optional ligand in the absorber is described in Section (I)(a)(iii).

(b) Reaction Conditions

The reaction conditions for the preparation of the liquid phase in the absorber are described above in Section (I)(a)(iv). The reaction conditions for the preparation of the liquid phase in the reaction vessel is described (I)(b)(iv).

(c) Output from Process

In a preferred embodiment, the process produces 1,1,1,3,3-pentachloropropane. In general, the process produces 1,1,1,3,3-pentachloropropane in at least 50 weight percent (wt %) in the liquid phase of the reactor. In various embodiments, 1,1,1,3,3-pentachloropropane is produced in at least 50 wt %, in at least 60 wt %, in at least 70 wt %, in at least 80 wt %, in at least 90 wt %, in at least 95 wt %, or in at least 99 wt % in the liquid phase of the reactor.

In general, carbon tetrachloride is converted into 1,1,1,3,3-pentachloropropane in at least 50% conversion. In various embodiments, the % conversion of carbon tetrachloride into 1,1,1,3,3-pentachloropropane is at least 50%, in at least 60%, in at least 70%, in at least 80%, in at least 90%, or at least 95%.

Generally, the process produces 1,1,1,3,3-pentachloropropane, light by-products, and heavy by-products. These heavy by-products are produced in less than 5 weight % in the entire product distribution. In various embodiments, these heavy by-products may be less than 4 weight %, less than 3 weight %, less than 2 weight %, or less than 1 weight %.

(d) Separation of 1,1,1,3,3-Pentachloropropane.

The separation of 1,1,1,3,3-pentachloropropane and the recycle streams is described above in Section (II).

Product effluent stream (b) comprising the 1,1,1,3,3-pentachloropropane produced in the process may have a yield of at least about 20%. In various embodiments, the product effluent stream (b) comprising 1,1,1,3,3-pentachloropropane produced in the process may have a yield of at least about 30%, at least about 50%, at least about 70%, at least about 75%, at least about 80%, at least about 85%, at least about 90%, at least about 95%, or at least about 99%.

The 1,1,1,3,3-pentachloropropane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (V) Preferred Embodiments: 1,1,1,3,3,3-Hexachloropropane

(a) Process for the Preparation of 1,1,1,3,3,3-Hexachloropropane

One aspect of the present disclosure encompasses processes for the preparation of 1,1,1,3,3,3-hexachloropropane. The process commences by preparing a liquid phase in the absorber comprising vinylidene chloride contacting carbon tetrachloride which does not contain any catalytic species. The liquid phase from the absorber is transferred to the reaction vessel comprising at least one metallic solid catalyst in the form of fixed bed of structured or unstructured packing or a powder capable of initiating the reaction of vinylidene chloride with carbon tetrachloride, at least one ligand, and optionally carbon tetrachloride under the reaction conditions described above. To be clear, species capable of initiating the reaction of vinylidene chloride with a halogenated methane comprising at least one chlorine atom are present in the reaction vessel and not in the absorber. The at least one metallic solid catalyst utilized in the second reaction vessel is described in Section (I)(b)(i). The optional ligand in the absorber is described in Section (I)(a)(iii).

(b) Reaction Conditions

The reaction conditions for the preparation of the liquid phase in the absorber are described above in Section (I)(a)(iv). The reaction conditions for the preparation of the liquid phase in the reaction vessel is described (I)(b)(iv).

(c) Output from Process

In a preferred embodiment, the process produces 1,1,1,3,3,3-hexachloropropane. In general, the process produces 1,1,1,3,3,3-hexachloropropane in at least 50 weight percent (wt %) in the liquid phase of the reactor. In various embodiments, 1,1,1,3,3,3-hexachloropropane is produced in at least 50 wt %, in at least 60 wt %, in at least 70 wt %, in at least 80 wt %, in at least 90 wt %, in at least 95 wt %, or in at least 99 wt % in the liquid phase of the reactor.

In general, carbon tetrachloride is converted into 1,1,1,3,3,3-hexachloropropane in at least 50% conversion. In various embodiments, the % conversion of carbon tetrachloride into 1,1,1,3,3,3-hexachloropropane is at least 50%, in at least 60%, in at least 70%, in at least 80%, in at least 90%, or at least 95%.

Generally, the process produces 1,1,1,3,3,3-hexachloropropane, light by-products, and heavy by-products. These heavy by-products are produced in less than 5 weight % in the entire product distribution. In various embodiments, these heavy by-products may be less than 4 weight %, less than 3 weight %, less than 2 weight %, or less than 1 weight %.

(d) Separation of 1,1,1,3,3,3-Hexachloropropane.

The separation of 1,1,1,3,3,3-hexachloropropane and the recycle streams is described above in Section (II).

Product effluent stream (b) comprising the 1,1,1,3,3,3-hexachloropropane produced in the process may have a yield of at least about 20%. In various embodiments, the product effluent stream (b) comprising 1,1,1,3,3,3-hexachloropropane produced in the process may have a yield of at least about 30%, at least about 50%, at least about 70%, at least about 75%, at least about 80%, at least about 85%, at least about 90%, at least about 95%, or at least about 99%.

The 1,1,1,3,3,3-hexachloropropane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (VI) Further Reaction of the Halogenated Alkanes

In one aspect, disclosed herein are processes for the conversion of halogenated alkanes, such as 1,1,1,3-tetrachloropropane, 1,1,1,3,3-pentachloropropane, or 1,1,1,3,3,3-hexachloropropane, to one or more hydrofluoroolefins. These processes comprise contacting the halogenated alkanes with a fluorinating agent in the presence of a fluorination catalyst, in a single reaction or two or more reactions. These processes can be conducted in either gas phase or liquid phase with the gas phase being preferred at temperatures ranging from 50° C. to 400° C.

Generally, a wide variety of fluorinating agents can be used. Non-limiting examples of fluorinating agents include HF, F₂, CIF, AlF₃, KF, NaF, SbF₃, SbF₅, SF₄, or combinations thereof. The skilled artisan can readily determine the appropriate fluorination agent and catalyst. Examples of hydrofluoroolefins that may be produced utilizing these processes include, but are not limited to 2,3,3,3-tetrafluoroprop-1-ene (HFO-1234yf), 1,3,3,3-tetrafluoroprop-1-ene (HFO-1234ze), 3,3,3-trifluoroprop-1-ene (HFO-1243zf), e-1-chloro-2,3,3,3-tetrafluoropropene (HCFO-1224yd), and 1-chloro-3,3,3-trifluoroprop-1-ene (HFCO-1233zd).

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “Tet” refers to carbon tetrachloride.

The term “TBP” refers to tributyl phosphate.

Having described the invention in detail, it will be apparent that modifications and variations are possible without departing from the scope of the invention defined in the appended claims.

### EXAMPLES

The following examples illustrate various embodiments of the invention.

### Example 1: Preparation 1,1,1,3-Tetrachloropropane (250 FB)

A 7.6 L reactor was constructed of Monel (R-1 in Table 1). To the bottom was added carbon steel packing with a porosity of about 76% and a total surface area of about 4.2 1/cm. To the top was added about 3 liters of 0.25-inch Monel Pro-Pak packing. CCl₄ containing 0.65 wt. % TBP was fed to the reactor at a feed rate to give an overall residence time of 6 hours. Ethylene was added to maintain a pressure of 9 barg. The temperature was controlled at 100° C. Liquid was circulated from the top of the reactor to the bottom at 320× of the CCl₄ fresh feed and liquid was withdrawn at a rate to control the level a little above the bed of the iron packing. The conversion of CCl₄ was found to be 83.8% and the selectivity to 250 FB was 94% as shown Run 1 in Table 1.

### Examples 2-7: Preparation of 1,1,1,3-Tetrachloropropane (250 FB)

In runs 2-7, the carbon steel packing was removed from R-1 and 78% of that packing were placed in a separate 3 L cylindrical chamber (R-2). In this setup the flow rate across R-1 and R-2 could be controlled independently. The absorption of ethylene took place in R-1, while the active complex generation occurred in R-2. The liquid level in R-1 was lowered to 20% such that the overall residence time was maintained. At similar conditions but with 22% less surface area the conversion (Run 2) was similar to the base case (Run 1). Run 3 shows that a lower circulation rate to R-2 significantly reduced conversion as compared to run 2. In contrast reducing the circulation rate thru R-1 (Run 4) had little impact on conversion as compared to run 2. Note: the conversion of run 4 as compared to run 2 slightly increased due to the increased circulation rate.

Runs 5 to 7 used approximately 4 times lower surface area than the base case. The conversion of Run 5 was lower compared to Run 2, which had the same flow rate but more Fe(0) surface area. Reducing the flow rate thru R-2 (Run 6) again showed a reduction in conversion. Increasing the liquid residence time by increasing the liquid level in R-1 to 50% confirmed that some reaction takes place in the bulk liquid outside the Fe(0) packing (Run 7).

TABLE 1
0.65% TBP, 9 barg, 100° C., residence time 6 h

| | Fe | R-1 | | | Conver- | Selec- |
|---|---|---|---|---|---|---|
| | Content | Level | Circulation | Circulation | sion | tivity |
| Run | % | % | Ratio R-1 | Ratio R-2 | % | % |
| 1 | 100 | 50 | 320 | 320 | 83.8 | 94.4 |
| 2 | 78 | 20 | 326 | 296 | 83.0 | 93.0 |
| 3 | 78 | 20 | 311 | 63 | 69.0 | 94.0 |
| 4 | 78 | 20 | 65 | 315 | 85.0 | 93.0 |
| 5 | 25 | 20 | 320 | 304 | 73.5 | 93.4 |
| 6 | 25 | 20 | 315 | 52 | 55.5 | 94.4 |
| 7 | 25 | 50 | 315 | 315 | 77.3 | 93.3 |

### Example 8: Using a ½-Inch Nozzle

Carbon tetrachloride containing 0.65 weight % TBP was fed to an absorber/reactor system at a rate of 3.1 kg/hr. A liquid circulation flow of 890 kg/h was pumped from the absorber bottom through a heat exchanger and a reactor, then back into the top of the absorber through a %-inch nozzle. The absorber was 4-inch diameter and 36-inch height and was maintained at about 50% liquid level. The top of the absorber above the liquid level was devoid of any packing. The gas phase of the absorber comprised ethylene, which was continuously fed to the absorber to maintain the pressure at 9.0 barg. The temperature of the circulating liquid was maintained at 90° C. The reactor was 4-inch diameter and 36-inch tall, and was packed with ¼-inch carbon steel rings. Liquid was continuously withdrawn from the system to control absorber level. The conversion of carbon tetrachloride in the withdrawn liquid was 78% and the selectivity to the desired 250 fb product was 95.8%. The jet mixing in the absorber was sufficient to achieve mass transfer of ethylene without additional mechanical agitation.

### Example 9: Using an Eductor Nozzle

Carbon tetrachloride containing 0.65 weight % TBP was fed to an absorber/reactor system at a rate of 3.0 kg/hr. A liquid circulation flow of 860 kg/h was pumped from the absorber bottom through a heat exchanger and a reactor, then back into the top of the absorber through a Schutte & Koerting Model 264 eductor equipped with a 3-mm orifice. The suction of the eductor pulled ethylene from the vapor space of the absorber and mixed it with the circulating liquid. The combined liquid and gas then flowed through a %-inch tail pipe before falling into the liquid contained in the absorber. The absorber was 4-inch diameter and 36-inch height and was maintained at about 50% liquid level. The top of the absorber above the liquid level was devoid of any packing. The gas phase of the absorber comprised ethylene, which was continuously fed to the absorber to maintain the pressure at 9.0 barg. The temperature of the circulating liquid was maintained at 100° C. The reactor was 4-inch diameter and 36-inch tall, and was packed with ¼-inch carbon steel rings. Liquid was continuously withdrawn from the system to control absorber level. The conversion of carbon tetrachloride in the withdrawn liquid was 80% and the selectivity to the desired 250 fb product was 96%. The jet mixing in the eductor/absorber combination was sufficient to achieve mass transfer of ethylene without additional mechanical agitation.

### Example 10: Preparation of 1,1,1,3,3-Pentachloropropane (250 Fa) without Using Structured Packing

Carbon tetrachloride containing 2.5 weight % TBP and FeCl₃:TBP mole ratio about 0.5 was fed to an absorber/reactor system at a rate of 3.1 kg/hr. A liquid circulation flow of 790 kg/h was pumped from the absorber bottom through a heat exchanger and a reactor, then back into the top of the absorber through a %-inch nozzle. The absorber was 4-inch diameter and 36-inch height and was maintained at about 50% liquid level. The top of the absorber above the liquid level was packed with ¼-inch Pro-Pak Monel packing. The gas phase of the absorber comprised vinyl chloride, which was continuously fed to the absorber to maintain the pressure at 1.5 barg. The temperature of the circulating liquid was maintained at 100° C. The reactor was 4-inch diameter and 36-inch tall, and was packed with ¼-inch carbon steel rings. Liquid was continuously withdrawn from the system to control absorber level. The conversion of carbon tetrachloride in the withdrawn liquid was 70% and the selectivity to the desired 240 fa product was 95.6%. The packed section in the absorber was sufficient to achieve mass transfer of vinyl chloride without additional mechanical agitation.

1. A process for preparing a halogenated alkane, the process comprising:
a) contacting, in an absorber,
at least one alkene, halogenated alkene, or combinations thereof with a liquid stream comprising a halogenated methane comprising at least one chlorine atom and optionally, a recycle stream,
to form a liquid phase,
the at least one alkene, halogenated alkene or combinations thereof being introduced to the absorber in a gas phase and wherein at least a portion of the at least one alkene or halogenated alkene fed to the absorber is a fresh material feed to the process;
b) transferring at least a portion of the liquid phase from the absorber into a reaction vessel to form a reaction mixture wherein the reaction mixture comprises at least one solid metallic catalyst; at least one alkene, halogenated alkene, or combinations thereof; at least one ligand, an optional recycle stream, or combinations thereof; a halogenated methane comprising at least one chlorine atom; and
c) forming a product mixture comprising the halogenated alkane, light by-products, and heavy by-products.

2. The process of claim 1, wherein the at least one metallic solid catalyst is in the form of at least one fixed bed of a solid packing, a powder, or combinations thereof.

3. The process of claim 2, wherein the fixed bed packing of a solid packing is a structured packing, unstructured packing, or combinations thereof.

4. The process of claim 1, wherein at least a portion of product effluent stream from the reaction vessel is recycled to the absorber wherein the product effluent stream comprises the halogenated alkane, light by-products, and heavy by-products.

5. The process of claim 1, wherein at least a portion of the reactor effluent stream is recycled back to the reactor wherein the reactor effluent stream comprises at least one alkene, halogenated alkene, or combinations thereof; at least one ligand, an optional recycle stream, or combinations thereof; a halogenated methane comprising at least one chlorine atom; the halogenated alkane, light by-products, and heavy by-products.

6. The process of claim 1, wherein a portion of the fresh material feeds comprising at least one alkene, halogenated alkene, or combinations thereof; at least one ligand, and a halogenated methane comprising at least one chlorine atom is added to the absorber, reaction vessel, or combinations thereof.

7. The process of claim 4, wherein the material being recycled to the absorber or the reactor has a recycle product effluent mass flow, while the fresh material feeds being added to the reactor has a fresh material feed mass flow, wherein the mass ratio of the recycle product effluent mass flow to the fresh material feed mass flow is adjusted to maintain the conversion of the process and/or to maintain the kinetics of the process.

8. The process of claim 1, wherein at least one product effluent stream, a portion of the reaction mixture effluent stream, or combinations thereof are sent to the absorber or the reaction vessel, wherein the temperature of the at least one product effluent stream, a portion of the reaction mixture effluent stream, or combinations thereof is maintained with a heat exchanger.

9. The process of claim 1, wherein the halogenated alkane is a chlorinated alkane.

10. The process of claim 9, wherein the chlorinated alkane is 1,1,1,3-tetrachloropropane (250 FB).

11. The process of claim 9, wherein the chlorinated alkane is 1,1,1,3,3-pentachloropropane (240 FA).

12. The process of claim 9, wherein the chlorinated alkane is 1,1,1,3,3,3-hexachloropropane.

13. The process of claim 1, wherein the halogenated methane comprising at least one chlorine atom comprises carbon tetrachloride.

14. The process of claim 1, wherein the alkene comprises ethylene.

15. The process of claim 1, wherein the halogenated alkene comprises vinyl chloride, vinylidene chloride, or combinations thereof.

16. The process of claim 1, wherein the at least one metallic solid catalyst comprises a metal, a metal powder, an alloy of a metal, or combinations thereof.

17. The process of claim 16, wherein the metal is selected from the group consisting of aluminum, bismuth, chromium, cobalt, copper, gallium, gold, indium, iron, lead, magnesium, manganese, mercury, nickel, platinum, palladium, rhodium, samarium, scandium, silver, titanium, tin, zinc, zirconium, and combinations thereof.

18. The process of claim 16, wherein the metal comprises iron metal, copper metal, an iron containing compound, a copper containing compound, an iron containing alloy, a copper containing alloy, or combinations thereof.

19. The process of claim 1, wherein the metal comprises iron metal, an iron containing compound, an iron containing alloy, or combinations of two or more thereof.

20. The process of claim 19, wherein the at least one metallic solid catalyst complexes to the at least one ligand to form an active catalytic species.

21. The process of claim 1, wherein the active catalytic species comprises Fe(0), Fe(II), Fe(III), or combinations thereof.

22. The process of claim 1, wherein the active catalytic species comprises Cu(0), Cu(I), Cu(II), or combinations thereof.

23. (canceled)

24. (canceled)

25. The process of claim 1, wherein the at least one ligand comprises at least one trialkylphosphate, at least one trialkylphosphite, an alkyl nitrile, or combinations thereof.

26-35. (canceled)

36. The process of claim 1, wherein the absorber, the reaction vessel, or combinations thereof are stirred.

37. The process of claim 1, wherein the absorber comprises a spray tower or packing to facilitate absorption and mixing of the reactants.

38. The process of claim 36, wherein the methods to stir the absorber and the reaction vessel independently comprise mechanical stirring, jet mixing, or combinations thereof.

39. The process of claim 38, wherein jet mixing of the absorber comprises at least one nozzle, at least one educting nozzle, or combinations thereof.

40. The process of claim 1, wherein the absorber further comprises a draft tube.

41. The process of claim 1, wherein the absorber further comprises rashig ring, pall rings for random packing, or combinations thereof.

42. The process of claim 1, wherein a portion of the reaction mixture effluent from the reaction vessel is recycled to the inlet of the absorber, to the inlet of the reaction vessel, or a combination of both inlets of the absorber and reaction vessel.

43-47. (canceled)

48. The process of claim 1, wherein the halogenated alkane is converted into a fluorinated product.

49. The process of claim 1, wherein the absorber is a packed column.

50. The process of claim 49 wherein the packed column comprises a fixed bed of structured or unstructured packing, or mixtures thereof.

51. A process for preparing a halogenated alkane, the process comprising:
providing a fresh material feed to an absorber, the fresh material feed comprising at least one alkene, halogenated alkene, a halogenated methane comprising at least one chlorine atom;
transferring at least a portion of a liquid phase from the absorber into a reaction vessel to form a reaction mixture wherein the reaction mixture comprises at least one solid metallic catalyst; at least one alkene, halogenated alkene, or combinations thereof; at least one ligand, an optional recycle stream, or combinations thereof; a halogenated methane comprising at least one chlorine atom.

52. The process of claim 46, wherein a recycle is mixed with the fresh material feed prior to providing to the absorber.
